(19) United States
(12) Patent Application Publication
(10) Pub. No.: US 2025/0046104 A1
(43) Pub. Date: Feb. 6, 2025

(54) SYSTEMS AND METHODS FOR DETECTING AND IDENTIFYING CELLS

(71) Applicant: Life Technologies Corporation, Carlsbad, CA (US)

(72) Inventors: Larry Rystrom, Carlsbad, CA (US); Kim Ippolito, Carlsbad, CA (US); Mary Kristina Hamilton, Carlsbad, CA (US); Nicolas Rognin, Carlsbad, CA (US)

(21) Appl. No.: 18/792,112

(22) Filed: Aug. 1, 2024

Related U.S. Application Data
(60) Provisional application No. 63/517,052, filed on Aug. 1, 2023.

(51) Int. Cl. G06V 20/69; G06T 7/00; G16H 30/40

(52) U.S. Cl. CPC G06V 20/69; G06T 7/0012; G16H 30/40; G06T 2207/30024

(57) ABSTRACT

Examples described herein provide systems and methods for quantifying cells. An example method includes receiving at least one image, improving a contrast of the at least one image to generate a contrast image, and performing a fit operation on the contrast image to generate a processed image. The method includes applying a filter to the processed image to generate a filtered image, identifying cells within the filtered image, and providing an output image including an indication of the cells.

### RELATED APPLICATIONS

This application claims the benefit of U.S. Provisional Patent Application No. 63/517,052, filed Aug. 1, 2023, the entire content of which is hereby incorporated by reference.

### FIELD

The present disclosure generally relates to microscope image analysis. More specifically, the present disclosure relates to detecting and identifying cells in microscope images.

### BACKGROUND

Cell viability counting is traditionally a manual, time-intensive process. Advances in the field of image processing have enabled imaging systems to automate some of these manual tasks or otherwise to reduce the amount of time and manual effort associated with determining cellular concentrations in a sample. Existing automated systems and methods for analyzing cell viability within a sample, however, suffer from many drawbacks. For example, many cell viability systems require use of dyes, labels, or other compounds to determine the viability of cells within a sample. The use of many of these compounds often require specialized, expensive, and/or bulky equipment to retrieve a readout of the results.

### SUMMARY

Examples described herein receive microscope image of cells as input and output a list of cell locations. The image of cells may be associated with Raman measurements, fluorescence measurements, or the like. To convert the image of cells to a quantitative description of a list of cells, example systems and methods include performing operations such as attribute morphology, exponential histogram fit for image auto-thresholding, connected component identification, watershed segmentation, and combinations thereof.

One example provides a method of identifying cells. The method includes receiving at least one image, improving a contrast of the at least one image to generate a contrast image, and performing a fit operation on the contrast image to generate a processed image. The method includes applying a filter to the processed image to generate a filtered image, identifying cells within the filtered image, and providing an output image including an indication of the cells.

Another examples provides one or more hardware storage devices storing instructions executable by one or more processing devices of an imaging system, the instructions including receiving at least one image, improving a contrast of the at least one image to generate a contrast image, and performing a fit operation on the contrast image to generate a processed image. The instructions include applying a filter to the processed image to generate a filtered image, identifying cells within the filtered image, and providing an output image including an indication of the cells.

Another example provides an imaging apparatus including a stage assembly operable to receive a cell counting slide, and a controller including an electronic processor and a memory. The controller is configured to capture the cell counting slide to generate at least one image, improve a contrast of the at least one image to generate a contrast image, and perform a fit operation on the contrast image to generate a processed image. The controller is configured to apply a filter to the processed image to generate a filtered image, identify cells within the filtered image, and provide an output image including an indication of the cells.

### BRIEF DESCRIPTION OF THE DRAWINGS

Features and advantages of the present technology will become more apparent from the following detailed description of example embodiments thereof taken in conjunction with the accompanying drawings in which:

FIG. 1 is a perspective view of an imaging system configured to perform one or more of the methods disclosed herein, in accordance with one or more embodiments of the present disclosure.

FIG. 2 is a flowchart of an example method performed by the imaging system of FIG. 1, in accordance with one or more embodiments of the present disclosure.

FIG. 3 is a block diagram of various example components within the imaging system of FIG. 1, in accordance with one or more embodiments of the present disclosure.

FIG. 4 is a flowchart of a cell viability count method performed by the imaging system of FIG. 1, in accordance with one or more embodiments of the present disclosure.

FIG. 5 is a flowchart of the cell viability count method of FIG. 4, in accordance with one or more embodiments of the present disclosure.

FIGS. 6A-6B provide an example input image captured by the imaging system of FIG. 1, in accordance with one or more embodiments of the present disclosure.

FIGS. 7A-7B provide an example increased contrast image of the input image of FIG. 6A, in accordance with one or more embodiments of the present disclosure.

FIGS. 8A-8D are histograms of the increased contrast image of FIG. 7A, in accordance with one or more embodiments of the present disclosure.

FIGS. 9A-9B provide an example auto-thresholded image of the increased contrast image of FIG. 7A, where the threshold value is automatically defined by histogram-fitting applied to the increased contrast image of FIG. 7A, and having highlighted areas of interest, in accordance with one or more embodiments of the present disclosure.

FIGS. 10A-10B provide an example filtered image of the auto-thresholded image of FIG. 9A, in accordance with one or more embodiments of the present disclosure.

FIGS. 11A-11B provide an image with identified and separated cell candidates within the filtered image of FIG. 10A, in accordance with one or more embodiments of the present disclosure.

FIGS. 12A-12B provide an output image including accepted cell candidates in the image of FIG. 11A, in accordance with one or more embodiments of the present disclosure.

While the present technology is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION

Unless otherwise defined, all technical and scientific terms used herein have the same meaning as commonly understood by one of ordinary skill in the art. In case of conflict, the present document, including definitions, will control. Example methods and systems are described below, although methods and systems similar or equivalent to those described herein can be used in practice or testing of the present disclosure. All publications, patent applications, patents and other references mentioned herein are incorporated by reference in their entirety. The systems, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise.

As used herein, the term “or” is intended to mean an inclusive “or” rather than an exclusive “or.” That is, unless specified otherwise, or clear from context, “X employs A or B” is intended to mean any of the natural inclusive permutations. That is, if X employs A, X employs B, or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form.

In addition, unless otherwise indicated, numbers expressing quantities, constituents, distances, or other measurements used in the specification and claims are to be understood as being modified by the term “about.” The terms “about,” “approximately,” “substantially,” or their equivalents, represent an amount or condition close to the specific stated amount or condition that still performs a desired function or achieves a desired result. For example, the terms “approximately,” “about,” and “substantially” may refer to an amount or condition that deviates by less than 10%, or by less than 5%, or by less than 1%, or by less than 0.1%, or by less than 0.01% from a specifically stated amount or condition.

The present disclosure is described with reference to the drawings, wherein like reference numerals are used to refer to like elements throughout. In the following description, for purposes of explanation, numbers of specific details are set forth in order to provide an improved understanding of the present disclosure. It may be evident, however, that the systems and methods of the present disclosure may be practiced without one or more of these specific details. In other instances, well-known structures and devices are shown in block diagram form to facilitate describing the systems and methods of the present disclosure. There is no specific requirement that a system, method, or technique relating to microscope image analysis include all of the details characterized herein to obtain some benefit according to the present disclosure. Thus, the specific examples characterized herein are meant to be example applications of the techniques described and alternatives are possible.

Cell viability counting was traditionally a manual, time-intensive process. Advances in the field of image processing have enabled imaging systems to automate some of these manual tasks or otherwise to reduce the amount of time and manual effort associated with determining cellular concentrations in a sample, particularly with respect to ascertaining the proportional number of live/dead cells (or viability counting) within a given sample.

Existing automated systems and methods for analyzing cell viability within a sample, however, suffer from many drawbacks. For example, many cell viability systems require use of dyes, labels, or other compounds to determine the viability of cells within a sample. The use of many of these compounds often require specialized, expensive, and/or bulky equipment to retrieve a readout of the results. As such, the equipment is unlikely to be readily available and/or positioned within the lab space as to make it accessible or convenient to use. Also, systems and methods utilizing machine learning techniques require large training datasets and can be computationally expensive.

Accordingly, to address these and other issues, the systems and methods disclosed herein provide accurate and automatic conversion of an image of cells to a quantitative description of a list of cells. In this manner, cell viability counting is provided automatically and without the manual, time-intensive process.

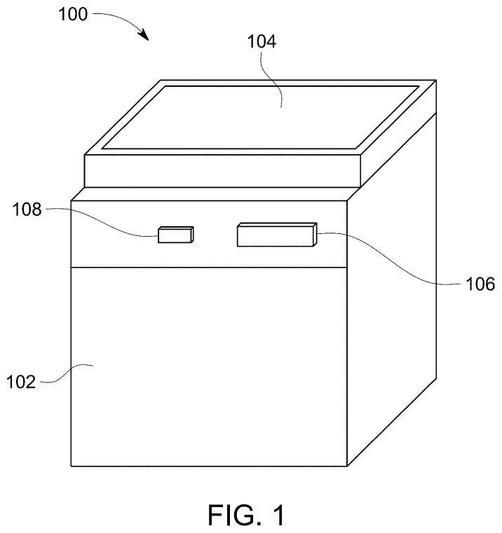

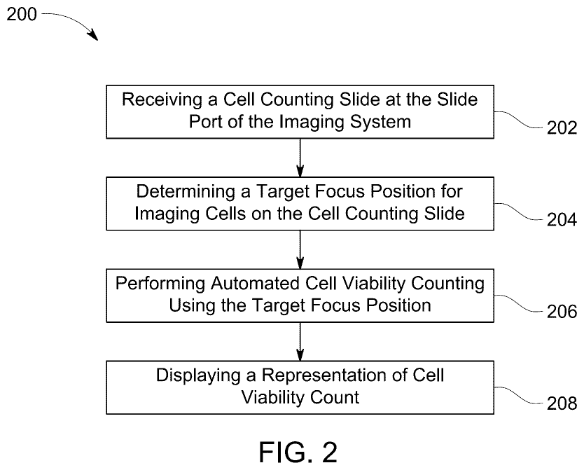

FIG. 1 is a perspective view of an imaging system 100 configured to perform one or more of the methods disclosed herein. The imaging system 100 of FIG. 1 is operable to facilitate the method associated with flow diagram 200 of automated cell viability count disclosed by the example flow diagram of FIG. 2. As shown, the imaging system 100 includes a housing 102, which encloses and protects the microscope and computing systems used for cell viability counts. The housing 102 includes a slide port/stage assembly 106 operable to receive a cell counting slide into the imaging system (at step 202). Once received therein, the imaging system 100 determines a target focus position for imaging cells on the cell counting slide (at step 204) and performs automated cell viability counting using the target focus position (at step 206). A representation of the cell viability count is displayed (at step 208) at the imaging system 100 using, for example, display 104. This representation and/or other data associated with the automated cell viability count can be removed from the imaging system 100 and/or saved to a separate device through user interaction with the communications module 108, which in some embodiments can include a USB port or other data exchange port known in the art.

One will appreciate, in view of the present disclosure, that the principles described herein may be implemented utilizing any suitable imaging system and/or any suitable imaging modality. The specific examples of imaging systems and imaging modalities discussed herein are provided by way of example and as a means of describing the features of the disclosed embodiments. Thus, the embodiments disclosed herein are not limited to any particular microscopy system or microscopy application and may be implemented in various contexts, such as brightfield imaging, fluorescence microscopy, flow cytometry, confocal imaging (e.g., 3D confocal imaging, or any type of 3D imaging), and/or others. For example, principles discussed herein may be implemented with flow cytometry systems to provide or improve cell counting capabilities. As another example, cell count and/or viability data obtained in accordance with techniques of the present disclosure may be used to supplement fluorescence data to improve accuracy in distinguishing among different cells.

Furthermore, one will appreciate, in view of the present disclosure, that any number of principles described herein may be implemented in various fields. For example, a system may implement the cell counting techniques discussed herein without necessarily also implementing techniques such as cell viability determination.

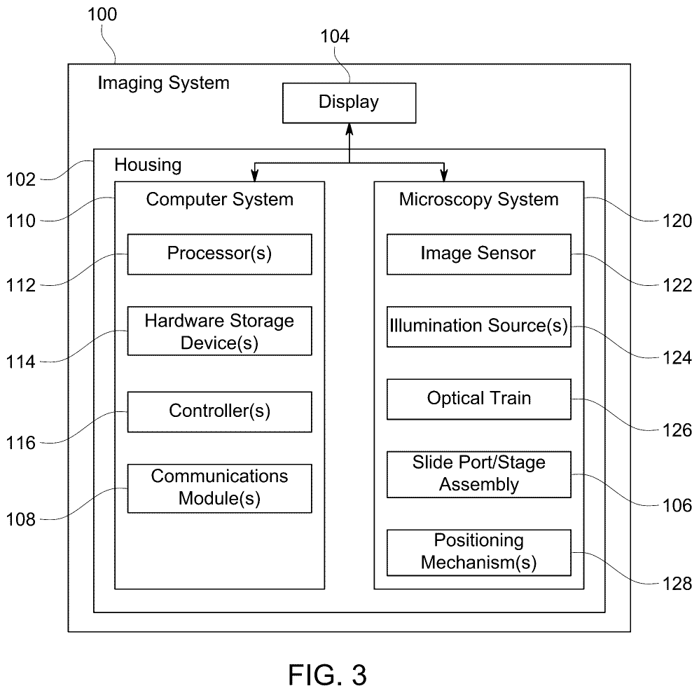

FIG. 3 is a schematic of various example components within the imaging system 100 of FIG. 1, in accordance with one or more embodiments of the present disclosure. As illustrated in FIG. 3, the imaging system 100 may include a computer system 110 and a microscopy system 120 included therewith. FIG. 3 conceptually represents the computer system 110 and the microscopy system 120 as disposed within the housing 102 of the imaging system 100. However, one will appreciate, in view of the present disclosure, that any portion of the computer system 110 or the microscopy system 120 may be disposed at least partially outside of the housing 102 within the scope of the disclosed embodiments.

FIG. 3 shows that the computer system 110 of the imaging system 100 can comprise various components, such as electronic processor(s) 112, hardware storage device(s) 114, controller(s) 116, and communications module(s) 108.

The electronic processor(s) 112 may comprise one or more sets of electronic circuitry that include any number of logic units, registers, and/or control units to facilitate the execution of computer-readable instructions (e.g., instructions that form a computer program). Such computer-readable instructions may be stored within the hardware storage device(s) 114, which may comprise physical system memory and which may be volatile, non-volatile, or some combination thereof.

The controller(s) 116 may comprise any suitable software components (e.g., set of computer-executable instructions) and/or hardware components (e.g., an application-specific integrated circuit, or other special-purpose hardware component(s)) operable to control one or more physical apparatuses of the imaging system 100, such as portions of the microscopy system 120 (e.g., the positioning mechanism(s) 128).

The communication module(s) 108 may comprise any combination of software or hardware components that are operable to facilitate communication between on-system components/devices and/or with off-system components/devices. For example, the communications module(s) 108 may comprise ports, buses, or other physical connection apparatuses for communicating with other devices (e.g., USB port, SD card reader, and/or other apparatus). Additionally, or alternatively, the communications module(s) 108 may comprise systems operable to communicate wirelessly with external systems and/or devices through any suitable communication channel(s), such as, by way of non-limiting example, Bluetooth, ultra-wideband, WLAN, infrared communication, and/or others.

As shown in FIG. 3, the imaging system 100 includes a microscopy system 120 having an image sensor 122, an illumination source 124, an optical train 126, the slide port/stage assembly 106 for receiving the sample slide, and a positioning mechanism 128.

The image sensor 122 is positioned in the optical path of the microscopy system and configured to capture images of the samples, which will be used in the disclosed methods to identify a target focus position and subsequently for performing automated cell viability counting. As used herein, the term “image sensor” or “camera” refers to any applicable image sensor compatible with the apparatuses, systems and methods described herein, including but not limited to charge-coupled devices, complementary metal-oxide-semiconductor devices, N-type metal-oxide-semiconductor devices, Quanta Image Sensors, combinations of the foregoing such as scientific complementary metal-oxide-semiconductor devices, and the like.

The optical train 126 may include one or more optical elements configured to facilitate viewing of the cell counting slide by directing light from the illumination source 124 toward the received cell counting slide. The optical train 126 may also be configured to direct light scattered, reflected, and/or emitted by a specimen within the cell counting slide toward the image sensor 122. The illumination source 124 may be configured to emit various types of light, such as white light or light of one or more particular wavelength bands. For example, the illumination source 124 can include a light cube, which may be installed and/or exchanged within the housing to any of a desired set of illumination wavelengths.

The positioning mechanism 128 can include any of an x-axis motor, a y-axis motor, and a z-axis motor that are operable to adjust the components of the optical train 126 and/or image sensor 122, accordingly.

FIG. 3 furthermore illustrates that, in some instances, the imaging system 100 includes a display 104. FIG. 3 indicates that the display 104 may be in communication, whether directly or indirectly, with various other components of the imaging system 100, such as the computer system 110 or the microscopy system 120 thereof (e.g., as indicated in FIG. 3 by the tri-headed arrow). For example, the imaging system may capture images using components of the microscopy system 120 and captured images may be processed and/or stored using components of the computer system 110 (e.g., electronic processor(s) 112, hardware storage device(s) 114, etc.), and the processed and/or stored images may be displayed on the display 104 for observation by one or more users.

As described herein, the components of the imaging system 100 may facilitate cell viability counting on the sample contained on the cell counting slide. In some instances, a representation of results of cell viability counting may be displayed on the display 104 of the imaging system 100 within a short time period after initiating cell viability counting processing for a cell counting slide inserted into the imaging system 100 (e.g., within a time period of about 20 seconds or less, or within about 10 seconds or less).

One will appreciate, in view of the present disclosure, that an imaging system may comprise additional or alternative components relative to those shown and described with reference to FIG. 3, and that such components may be organized and/or distributed in various manners.

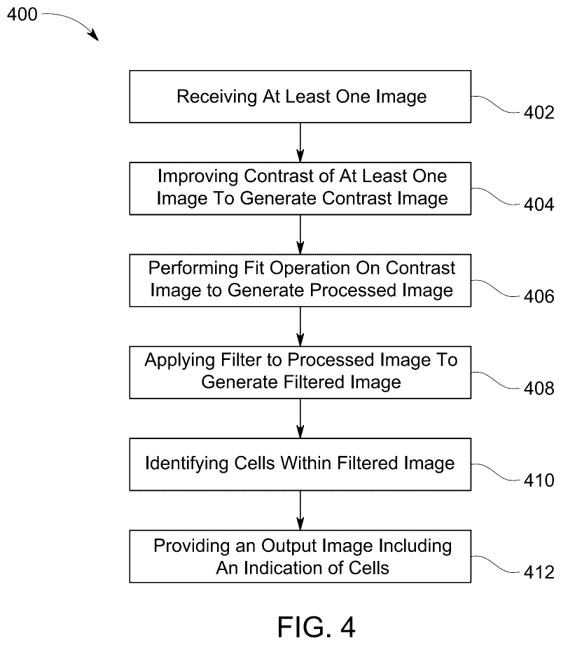

FIG. 4 is a flowchart of an example method 400 for performing automated cell viability counting. The method 400 may be performed, for example, by the controller(s) 116, the electronic processor(s) 112, or a combination thereof.

The method 400 includes receiving an at least one image (for example, an input image) (at step 402). For example, the image sensor 122 captures an image of a sample. In another example, an image is acquired from, for example, a server or a memory device. Accordingly, the image may be a previously-captured image.

The method 400 includes improving the contrast of the at least one image to generate a contrast image (at step 404) by, for example, performing area attribute operations (e.g., area attribute opening, area attribute closing), greyscale operations, and the like.

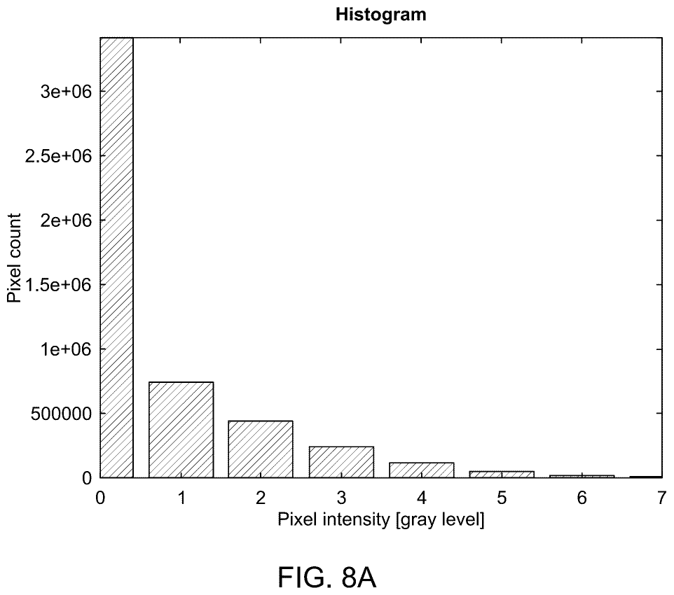

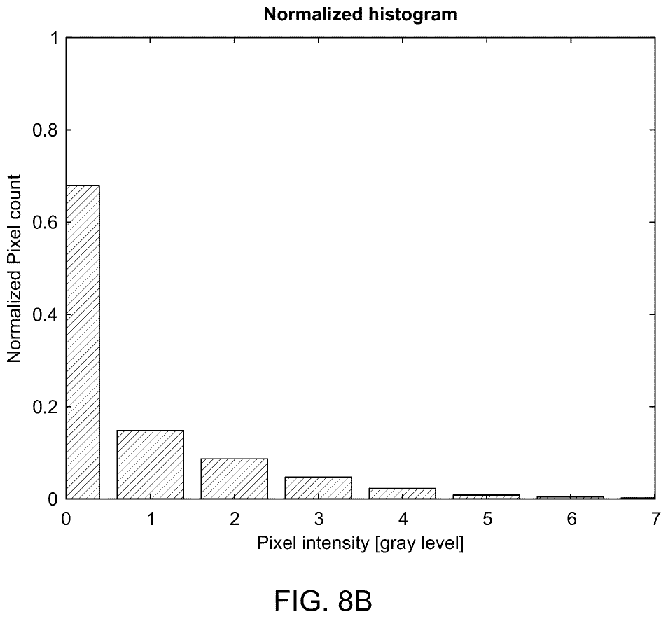

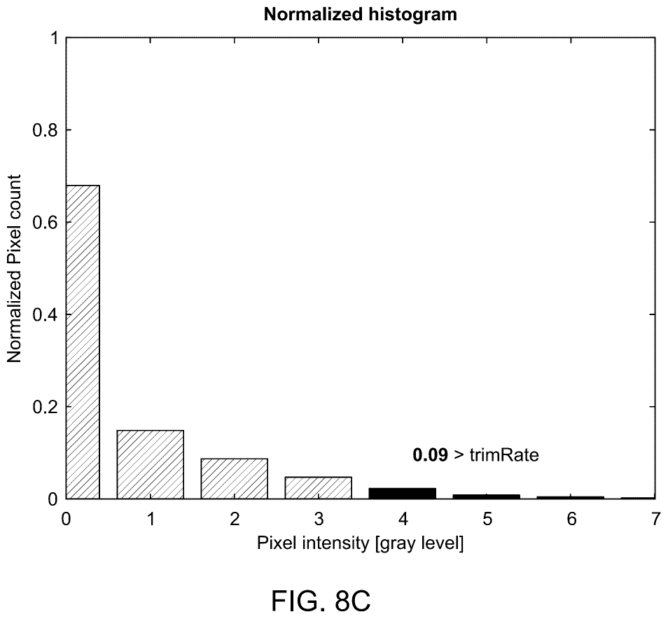

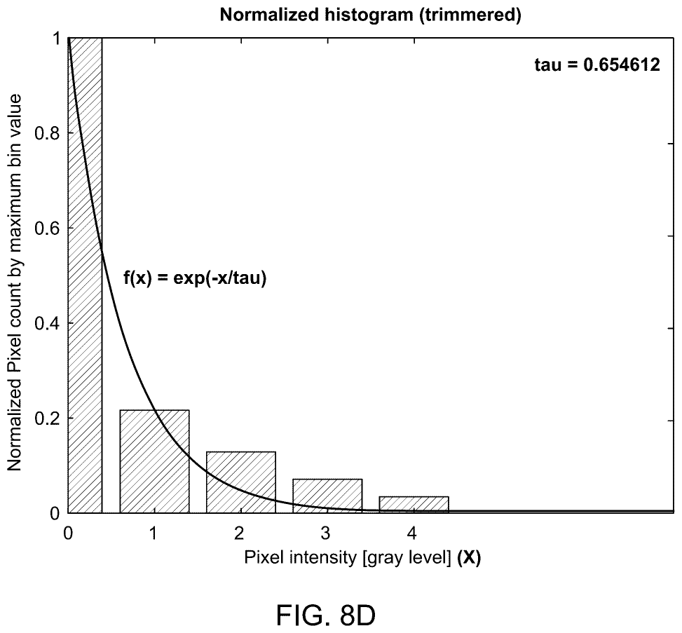

The method 400 includes performing a fit operation on the contrast image to generate a processed image (at step 406). For example, as described below in more detail with respect to FIGS. 8A-8D, an exponential histogram fit operation may be performed on the contrast image (e.g., the improved-contrast image). Other fit operations may alternatively be performed, such as a curve fit operation. In another example, a Gaussian function may be implemented for the histogram fit operation, as defined by Equation (1):

$$f ( x ) = \frac{1}{σ \sqrt{2 π}} {e}^{- {( x - μ )}^{2} / ( 2 {σ}^{2} )} \qquad Equation ( 1 )$$

Where:
σ is the standard deviation; and
μ is the weighted average.

The method 400 includes applying a filter to the processed image to generate a filtered image (at step 408). For example, one or more of a binary opening operation followed by a binary closing operation with square kernel of 2×2 pixels, a binary mask, a median filter, a low-pass filter, and the like may be applied to the third image to remove noise, trim tendrils, and fill small holes within the third image.

The method 400 includes identifying cells within the filtered image (at step 410). For example, a connected component operation (e.g., an eight-connected component operation) may be performed to identify cells within the filtered image, as described below in more detail.

The method 400 includes providing an output image including an indication of cells (at step 412). For example, a representation of the cells within the input image is displayed at the imaging system 100 using, for example, display 104. In this manner, cell viability count is performed.

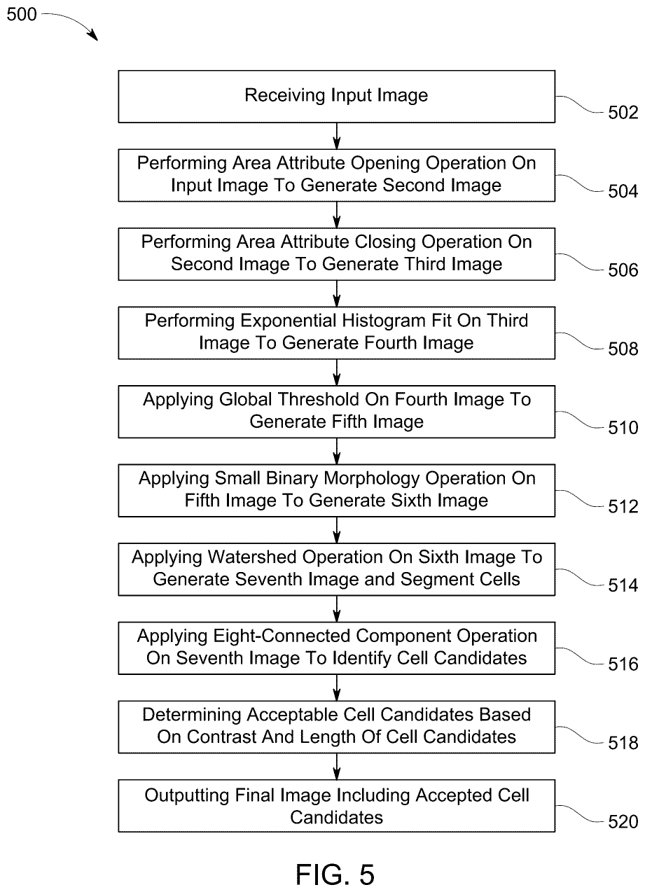

FIG. 5 is a flowchart of one example implementation of the method 400 for performing automated cell viability counting. The method 500 may be performed, for example, by the controller(s) 116, the electronic processor(s) 112, or a combination thereof.

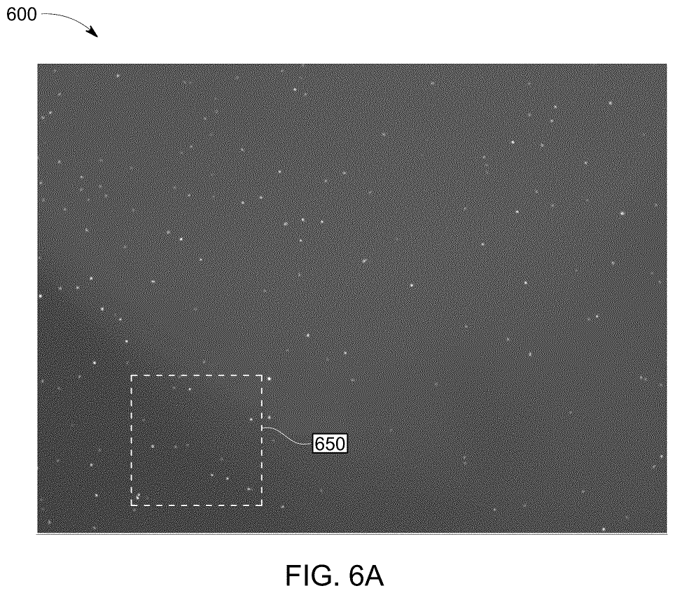

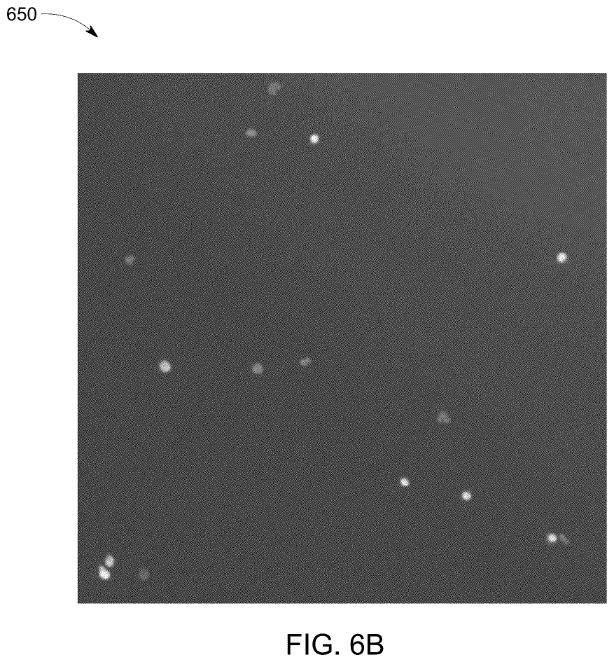

The method 500 includes receiving an input image (at step 502). For example, the image sensor 122 captures an image of a sample. FIG. 6A illustrates an example input image 600 discussed with respect to the method 500. FIG. 6B illustrates a highlighted portion 650 of the input image 600 in greater detail. The input image 600 may be received by the controller(s) 116.

The method 500 includes performing an area attribute grayscale opening operation on the input image 600 to generate a second image (at step 504). In some implementations, the area attribute opening is performed with a radius of size between 7.4 μm and 7.8 μm. In some implementations, the area attribute opening is performed with a radius of size 7.6 μm.

The method 500 includes performing an area attribute grayscale closing operation on the second image to generate a third image (at step 506). In some implementations, the area attribute closing is performed with an area between 4 pixels and 6 pixels. In some implementations, the area attribute closing is performed with an area of 5 pixels.

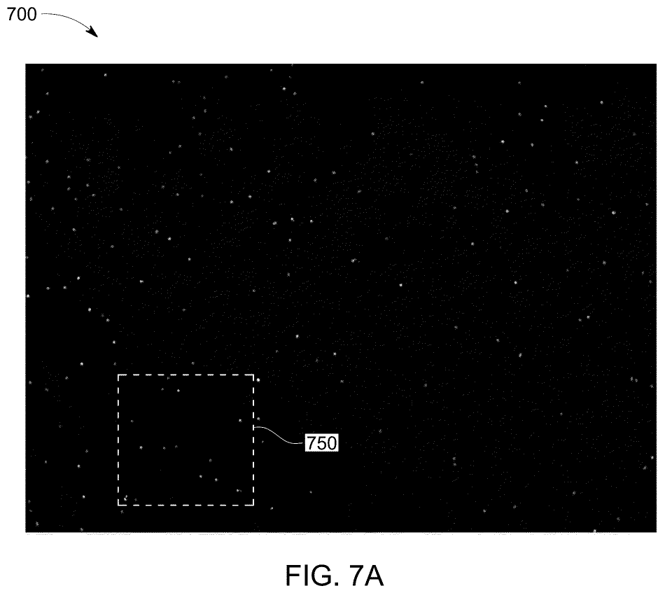

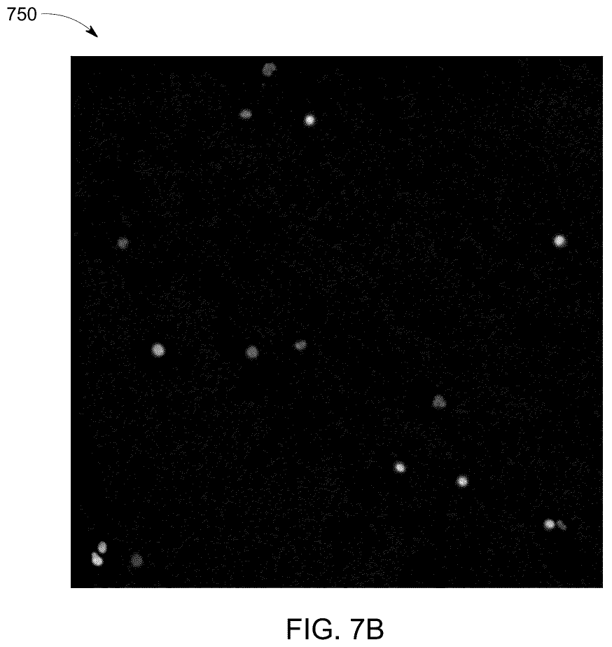

Step 504, step 506, or the combination thereof increases the contrast of the input image 600. For example, FIG. 7A illustrates an example third image 700. FIG. 7B illustrates a highlighted portion 750 of the third image 700 in greater detail. The third image 700 has increased contrast between the background and foreground compared to the input image 600.

Returning to FIG. 5, the method 500 includes performing an exponential histogram fit operation on the third image 700 to generate a fourth image (at step 508). For example, FIGS. 8A-8D illustrate histograms representing the third image 700 during a histogram fit operation. The histograms illustrate the relationship between pixel count and pixel intensity. FIG. 8A illustrates a histogram of the third image 700 counting numbers of pixels at each of a plurality of pixel intensity values (grey scale). FIG. 8B illustrates the histogram of FIG. 8A normalized by total pixel count (i.e., divide the number of pixels for each intensity value by the total number of pixels). FIG. 8C illustrates the normalized histogram of FIG. 8B with identified bin values greater than a trim rate. FIG. 8D illustrates the histogram of FIG. 8C with an overlaid exponential function and normalized by maximum bin value. The exponential function is provided by

$${e}^{- \frac{x}{t a u}} ,$$

where tau is the fitting parameter of the exponential function. Tau may be empirically defined for each individual histogram. In the example of FIG. 8D, the tau value is 0.654612.

In some implementations, the log trim rate is between a value of −1 and −2. In the examples of FIGS. 8A-8D, the log trim rate is −1.3. Accordingly, to identify the trim rate:

$$LogTrimRate = - 1.3$$

$$TrimRate = {10}^{L o g T r i mRate} = 1 {0}^{- 1.3} \approx 0.05$$

The trim rate limits the bins used in the analysis to a select number of top bins (i.e., intensity values having the highest number of counts) in the histogram. For example, in the above example, a trim rate of 0.05 indicates that 5% of the pixels in the lowest bins should be trimmed. Accordingly, to apply the trim rate, the normalized number of pixels in each bin (each intensity value) starting from the smallest bin (i.e., the intensity value with the smallest normalized count) is added until the sum exceeds the trim rate. As illustrated for the example bins in FIG. 8C, the normalized number of pixels starting with bin 7 are summed. The sum of bins 7, 6, and 5 is less than the trim rate (0.05), but the sum of bins 7, 8, 5, and 4 (0.09) is greater than the trim rate (0.05). Thus, in this example, bins 1, 2, 3, and 4 are retained but bins 7, 6, and 5 are trimmed (ignored). As illustrated in FIG. 8D, the retained bins are normalized by dividing each retained bin by the maximum value among the retained bins before performing the exponential histogram fit operation. In some implementations, the exponential histogram fit operation has a log false-alarm rate of between −1 and −7. In the examples of FIGS. 8A-8D, the log false-alarm rate is −4.2. The log false-alarm rate is used to determine a global threshold value:

$$LogF a l s e A l a r mRate = - 4.2$$

$$FalseAlarmRate == 1 {0}^{L o g F a l s e A l a r mRate}$$

$$global threshold = - tau * ln ( FalseAlarmRate )$$

In the example of FIGS. 8A-8D, where tau=0.654612, the global threshold is 6.3307.

Automatic thresholding by histogram fitting provides overall robustness as compared to other methodologies. Histogram fitting is unique for each input image, and accordingly the global threshold is adapted for each input image.

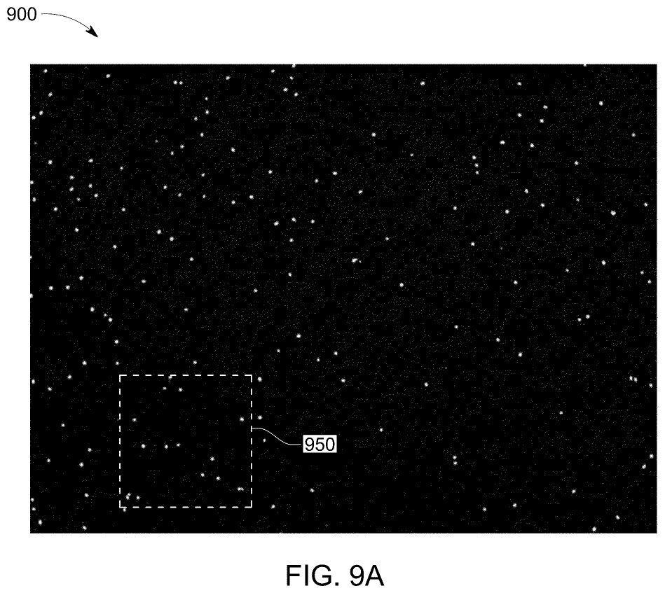

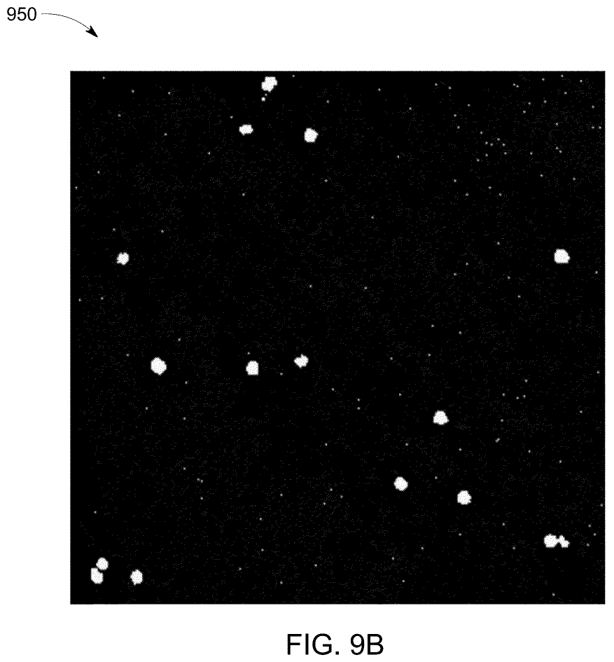

Returning to FIG. 5, the method 500 includes applying the global threshold on the fourth image to generate a fifth image (at step 510). For example, the global threshold may be applied to apply a binary mask to highlight areas of interest within the fourth image. In other words, every pixel having a value above the threshold to set to one value and all other pixels are set to a different value. FIG. 9A illustrates an example fifth image 900 including highlighted areas of interest. FIG. 9B illustrates a highlighted portion 950 of the fifth image 900 in greater detail

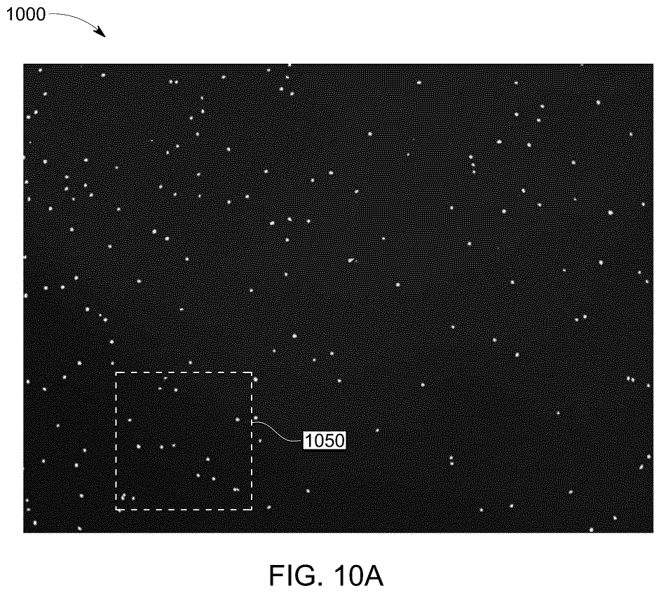

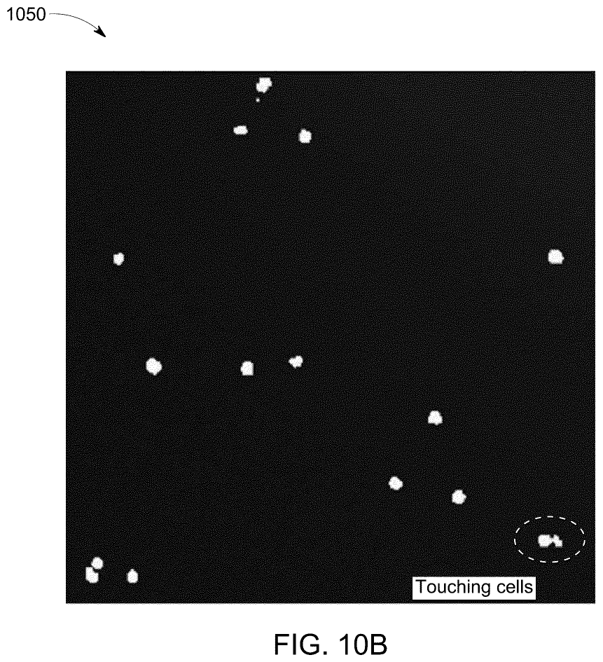

As illustrated in FIG. 5, the method 500 includes applying a small binary morphology operation on the fifth image 900 to generate a sixth image (at step 512). For example, a small binary morphology operation may be performed to remove background noise within the fifth image 900, as well as trim tendrils around the areas of interest and fill small holes within the fifth image 900. FIG. 10A illustrates an example sixth image 1000 following the small binary morphology operation. FIG. 10B illustrates a highlighted portion 1050 of the sixth image 1000 in greater detail. Example of binary morphology operation includes a morphological opening followed by a morphological closing with square kernel of 2×2 pixels for both opening and closing operators.

The small binary morphology operation assists with removing foreground holes. Foreground holes may appear in images based on the appearance of cells from different focus positions (e.g., the different z-height focus positions from which the image is captured by image sensor 122). Thus, in some instances, foreground hole fill operations may be performed to expand connected pixels in a way that fills the foreground holes. In some instances, after performing morphological operations on the image (e.g., the small binary morphology operation), the imaging system 100 may define the connected components within the image in preparation for additional processing.

The method 500 includes applying a watershed operation on the sixth image 1000 to generate a seventh image and segment cell candidates (at step 514). For example, two or more cells within the sixth image 1000 may be touching. The watershed operation may be implemented to identify and logically separate the touching cells.

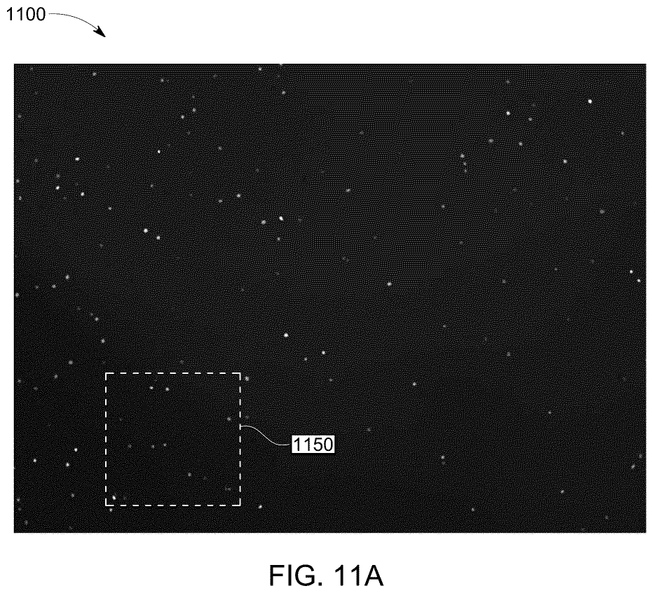

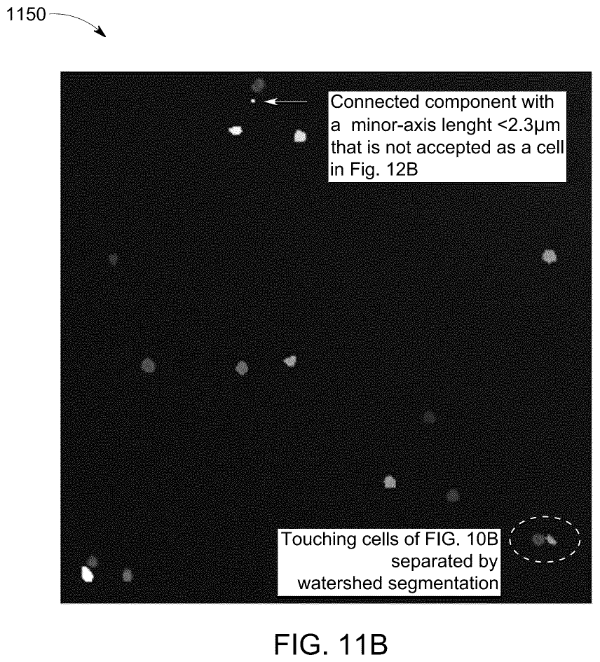

The method 500 includes applying an eight-connected component operation on the seventh image to identify cell candidates (at step 516). For example, connected components within the seventh image may be identified to label candidate cells within the seventh image. In some implementations, a four-connected component operation is performed. FIG. 11A illustrates an example seventh image 1100 including identified cell candidates. FIG. 11B illustrates a highlighted portion 1150 of the seventh image 1100 in greater detail.

As used herein, “connectedness” as used in “connected components,” refers to which pixels are considered neighbors of a pixel of interest. A connected component is a set of pixels of a single value, for example, the value representing black, wherein a path can be formed from any pixel of the set to any other pixel in the set without leaving the set, for example, by traversing only black pixels. In general terms, a connected component may be either “four-connected” or “eight-connected.” In the four-connected case, the path can move in only horizontal or vertical directions, so there are four possible directions. In the eight-connected case, the path between pixels may also proceed diagonally.

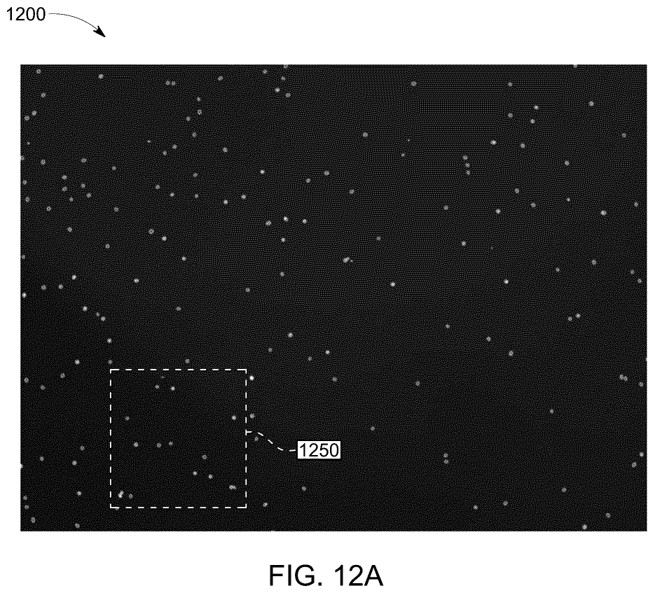

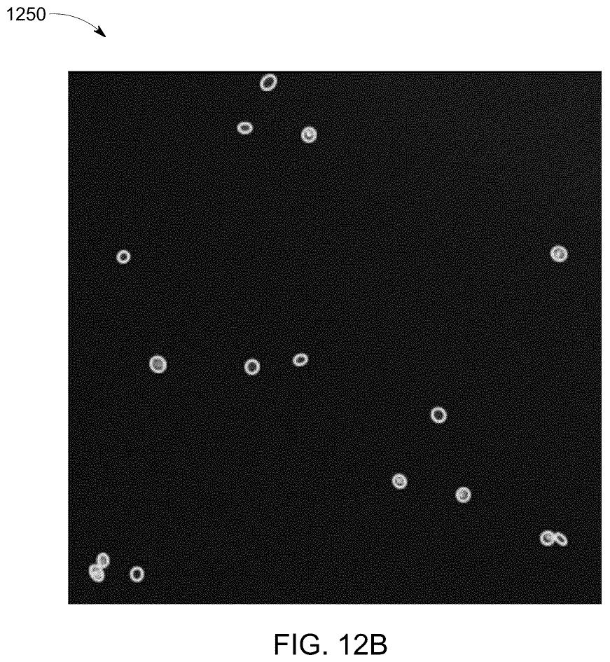

The method 500 includes determining acceptable cell candidates based on the contrast and length of the cell candidates (at step 518). For example, the contrast of the cell candidates and the length of the cell candidates can be compared to a contrast threshold and a length threshold, respectively. The contrast threshold may be, for example, between 15 and 25 bytes. The length threshold may be, for example, a minor-axis length between 1.5 μm and 3.0 μm. FIG. 12A illustrates an eighth image 1200 including accepted cell candidates. FIG. 12B illustrates a highlighted portion 1250 of the eighth image 1200 in greater detail. In the example of FIG. 12A, the contrast threshold is 20 bytes, and the length threshold is 2.3 μm.

The method 500 includes outputting an image (e.g., the eighth image 1200) including the accepted cell candidates (at step 520). For example, an indication or representation of the cells within the input image is displayed (e.g., by the controller(s) 116) at the imaging system 100 using, for example, display 104. In this manner, cell viability count is performed. Cells may be identified by being highlighted, circled, indicated with an ellipse, or the like. The ellipses may be fit to each connected component defined within the eighth image 1200. In some instances, the controller(s) 116 provide data regarding the accepted cell candidates on the display 1104, such as an ellipse center coordinate (in pixels), an ellipse semi-major axis (in pixels), an ellipse semi-minor axis (in pixels), an ellipse angle (in degrees), cell viability (for example, whether cells are dead or alive), cell brightness (for example, average grayscale intensity within the ellipse), circularity of the cells (for example, ranging from 0 to 1, where 1 is a perfect circle), and the like. It should be understood that the identified cells and associated cell count information may be output in various formats and forms. For example, information regarding identified cells may be presented graphically (e.g., as generally illustrated in FIGS. 12A-12B), textually, or a combination thereof.

As described above in the detailed description, reference is made to the accompanying drawings that form a part hereof wherein like numerals designate like parts throughout, and in which is shown, by way of illustration, implementations that may be practiced. It is to be understood that other implementations may be utilized, and structured or logical changes may be made, without departing from the scope of the present disclosure. Therefore, the detailed description as described above is not to be taken in a limiting sense.

Various operations may be described as multiple discrete actions or operations in turn, in a manner that is most helpful in understanding the subject matter disclosed herein. However, the order of description should be construed as to imply that these operations are necessarily order dependent. In particular, these operations may not be performed in the order of presentation. Operations described may be performed in a different order from the described implementation. Various additional operations may be performed, and/or described operations may be omitted in additional implementations.

What is claimed is:

1. A method of identifying cells implemented via one or more electronic processors, the method comprising:
receiving at least one image;
improving a contrast of the at least one image to generate a contrast image;
performing a fit operation on the contrast image to generate a processed image;
applying a filter to the processed image to generate a filtered image;
identifying cells within the filtered image; and
providing an output image including an indication of the cells.

2. The method of claim 1, wherein improving the contrast of the input image includes:
performing an area attribute opening operation on the input image to generate an intermediate image; and
performing an area attribute closing operation on the intermediate image to generate the contrast image.

3. The method of claim 2, wherein the area attribute opening operation is performed with a radius of size between 7.4 μm and 7.8 μm.

4. The method of claim 2, wherein the area attribute closing operation is performed with an area between 4 pixels and 6 pixels.

5. The method of claim 1, wherein performing the fit operation on the contrast image includes:
generating a histogram representation of the input image based on intensity values of pixels within the input image; and
normalizing the histogram representation using a total number of the pixels within the input image to generate a normalized histogram representation.

6. The method of claim 5, wherein performing the fit operation on the contrast image further includes:
trimming at least one bin included in the normalized histogram representation based on a trim rate to generate a trimmed histogram representation;
determining an exponential function that fits the trimmed histogram representation;
determining a global threshold value based on the exponential function; and
applying the global threshold value to the contrast image to generate the processed image.

7. The method of claim 1, wherein applying the filter to the processed image to generate the filtered image includes performing a small binary morphology operation on the processed image.

8. The method of claim 1, wherein identifying cells within the filtered image includes applying a watershed operation on the filtered image.

9. The method of claim 1, wherein identifying cells within the filtered image includes performing an eight-connected component operation on the filtered image.

10. A computer system configured to identify cells, comprising one or more electronic processors and one or more hardware storage devices having stored thereon computer-executable instructions that when executed by the one or more electronic processors configure the computer system to perform the method of claim 1.

11. One or more hardware storage devices storing instructions executable by one or more processing devices of an imaging system, the instructions including:
receiving at least one image;
improving a contrast of the at least one image to generate a contrast image;
performing a fit operation on the contrast image to generate a processed image;
applying a filter to the processed image to generate a filtered image;
identifying cells within the filtered image; and
providing an output image including an indication of the cells.

12. An imaging apparatus comprising:
a stage assembly operable to receive a cell counting slide; and
an electronic processor configured to:
capture the cell counting slide to generate at least one image;
improve a contrast of the at least one image to generate a contrast image;
perform a fit operation on the contrast image to generate a processed image;
apply a filter to the processed image to generate a filtered image;
identify cells within the filtered image; and
provide an output image including an indication of the cells.

13. The imaging apparatus of claim 12, wherein the electronic processor is configured to improve the contrast of the at least one image by:
performing an area attribute opening operation on the at least one image to generate an intermediate image; and
performing an area attribute closing operation on the intermediate image to generate the contrast image.

14. The imaging apparatus of claim 13, wherein the area attribute opening operation is performed with a radius of size between 7.4 μm and 7.8 μm.

15. The imaging apparatus of claim 13, wherein the area attribute closing operation is performed with an area between 4 pixels and 6 pixels.

16. The imaging apparatus of claim 12, wherein the electronic processor is configured to perform the fit operation on the contrast image by:
generating a histogram representation of the at least one image based on intensity values of pixels within the at least one image; and
normalizing the histogram representation using a total number of the pixels within the at least one image to generate a normalized histogram representation.

17. The imaging apparatus of claim 16, wherein the electronic processor is further configured to perform the fit operation on the contrast image by:
trim at least one bin included in the normalized histogram representation based on a trim rate to generate a trimmed histogram representation;
determine an exponential function that fits the trimmed histogram representation;
determine a global threshold value based on the exponential function; and
apply the global threshold value to the contrast image to generate the processed image.

18. The imaging apparatus of claim 12, wherein the electronic processor is configured to apply the filter to the processed image to generate the filtered image by performing a small binary morphology operation on the processed image.

19. The imaging apparatus of claim 12, wherein the electronic processor is configured to identify cells within the filtered image by applying a watershed operation on the filtered image.

20. The imaging apparatus of claim 12, wherein the electronic processor is configured to identify cells within the filtered image by performing an eight-connected component operation on the filtered image.
